(19) United States
(12) Patent Application Publication
(10) Pub. No.: US 2013/0039968 A1
(43) Pub. Date: Feb. 14, 2013

(54) MATERIALS AND PROCESSES FOR PRODUCING ANTITOXIC FABRICS

(71) Applicants: Pierre Jean Messier, Mirabel (CA); David Ohayon, Mirabel (CA)

(72) Inventors: Pierre Jean Messier, Mirabel (CA); David Ohayon, Mirabel (CA)

(73) Assignee: TRIOMED INNOVATIONS CORP., South Burlington, VT (US)

(21) Appl. No.: 13/580,265

(22) PCT Filed: Feb. 22, 2011
(86) PCT No.: PCT/US11/25734

§ 371 Date: Oct. 26, 2012

Related U.S. Application Data
(60) Provisional application No. 61/306,810, filed on Feb. 22, 2010.

(51) Int. Cl. D04H 3/013; A61K 33/18; A61K 33/40; D04H 1/00; A61K 33/00; A61P 39/00; A61P 31/00; A61K 9/70; A61K 33/20

(52) U.S. Cl. 424/445; 424/667; 424/616; 424/661; 424/723; 264/103; 442/408

(57) ABSTRACT

The invention provides a novel method of generating fabrics with outstanding antitoxic properties. The antitoxic properties are imparted to the fabric by introducing an active agent such as an antimicrobial or antiviral agent to the fabric. The active agent may be introduced into the fabric at multiple stages of the manufacturing process. For nonwoven fabrics, the active agent can be introduced during web formation and/or during post-processing steps. The fabrics produced in accordance with the present invention have widespread utility. For instance, they can be used as wound dressings, gowns, drapes, air filters, protective clothing and wipes.

### BACKGROUND OF INVENTION

The production of woven and nonwoven materials requires a series of processing steps. Nonwovens are generally produced by extruding a polymer melt, cooling the melt and then either generating (for example, spinning) the melt into a series of filaments of different diameters ranging from nanometers to millimeters or meltblown and spunbond materials. The filaments are then brought together to form a loose web and the fibers of the web are bonded (or entangled) together. The bonding process imparts strength and integrity to the web. There are multiple methods of conducting each of these steps which are highly dependent upon the nature of the desired end product.

Nonwoven products are often categorized based on the procedure by which they are formed. Generally, nonwovens can be dry formed or wet laid. Dry formed fibers include air laid fibers, dry laid (carded) fibers, spunbond fibers, meltblown fibers and electrospun fibers. The webs produced by these steps can be subjected to additional processing steps, such as bonding to impart strength, flexibility and other desired properties to the web. For instance, the web may be subjected to hydroentanglement or calendering.

One desirable property that may be incorporated into a woven or nonwoven material is antitoxin capacity. To achieve this end, an antitoxic agent is added to the material either during or after processing of the material. For instance, the antitoxic agent may be glued, sprayed, sublimated or inserted via dipping onto a fiber, or onto a woven or nonwoven material following production. Alternatively, the antitoxic agent may be added during processing, either early or late in the production. The method of incorporation of the antitoxic agent may have important consequences in imparting the desired efficacy and toxicology to the material.

In the case of nonwoven materials, various methods have been described for incorporating materials into the web. As mentioned above, post-processing procedures such as spraying and gluing antimicrobial agents to the surface of a nonwoven material are known in the art. Certain techniques of incorporating an antimicrobial agent in a fiber during processing are also known in the art. One such method involves physically entrapping the active agent within the three-dimensional structure of the nonwoven material. The active agent must have the appropriate size to be entrapped within the matrix structure of the nonwoven web. For instance, U.S. Publication No. 2006/0144403 (the '403 publication), to Messier, describes several methods of physically entrapping an active agent such as an iodine demand disinfectant resin in a three-dimensional nonwoven matrix. The '403 publication is hereby incorporated by reference in its entirety. Another method involves making use of a meltblown system where the desired active agent is provided in a cloud at the location closest to the extrusion point of the fibers. The cloud of active agent envelops the extruded fibers exiting a spinneret. Upon cooling, the active agent becomes physically entrapped within the fibers on the collecting web.

In addition to physically entrapping the active agent, the active agent may be incorporated directly into the fiber. Certain methods of incorporating an antimicrobial agent into a nonwoven material are also known in the art. Generally, the active agent is blended with the polymer prior to extrusion so that it is present throughout the polymer. Upon solidification of the polymer, the active agent is dispersed throughout the resultant fiber. The active agent may diffuse to the surface of the nonwoven, where it exerts its toxic effect on the microorganism/toxin. For example, the '403 publication describes a method in which polymer granules are placed in a hopper along with active agent in powder form, preferably an iodine/resin disinfectant, prior to extrusion. The two components are then heated, extruded and attenuated to form fibers having the active agent incorporated therein. The resulting fibers having the active agent embedded can be air laid, vacuum laid or water laid. Nonwoven materials generated from this process may be utilized in various applications.

### SUMMARY OF INVENTION

Although methods described above for entrapping antitoxic agents into the three-dimensional matrix or the fibers of a nonwoven web produce efficacious materials, it is found that significant loss of the antitoxic agent may be encountered during processing. In the meltblown procedure, for instance, it is found that the steps of heating and extrusion may result in sublimation or leeching of the antitoxic agent from the web. The same holds true for other downstream steps of the process. Methods of producing materials with higher concentrations of active antitoxic agent would result in more efficacious materials.

In accordance with these objectives, a new manufacturing process for generating woven and/or non-woven materials or fabrics with a high level of antitoxic (e.g., biocidal or chemical) activity has been developed. The novel manufacturing process significantly increases the amount of active antitoxic agent that can be incorporated into a fabric by introducing one or more antitoxic agents during multiple steps of the woven and/or non-woven material manufacturing process.

In one aspect, the invention is directed to a manufacturing process for producing antitoxic non-woven materials comprising the steps of forming a plurality of staple fibers comprising a polymer and an iodinated resin, wet laying said staple fibers in an aqueous solution comprising an additional active agent to generate a wet laid web, subjecting said wet laid web to hydroentangling or calendering, and isolating the fabric. In certain embodiments, the additional active agent comprises iodine, bromine, chlorine, metals and/or hydrogen peroxide. In certain embodiments, the additional active agent comprises a liquid, solid or gaseous mixture of iodine molecule with or without potassium iodine. In certain embodiments, the additional active agent is incorporated in the web during the hydroentangling or calendaring step.

In another aspect, the invention is directed to a manufacturing process for producing an antitoxic woven and/or nonwoven material comprising the steps of forming a plurality of staple fibers comprising a polymer and an iodinated resin, air laying said staple fiber in a chamber containing a gaseous active agent to generate a dry laid web, subjecting said dry laid web to hydroentangling or calendering, and isolating the material. In certain embodiments, the gaseous active agent comprises iodine, bromine, chlorine, metals, fluorine and/or hydrogen peroxide. In certain embodiments, an additional active agent is incorporated in the web during the hydroentangling or calendering step.

In yet another aspect, the invention is directed to a manufacturing process for producing an antitoxic nonwoven material comprising the steps of forming a plurality of staple fibers comprising a polymer and an iodinated resin, carding said staple fibers to form a carded web, subjecting the carded web to a solution containing an active agent, further subjecting the carded web to hydroentangling or calendering process, and isolating the material. In certain embodiments, the active agent comprises iodine, bromine, chlorine, metals, fluorine and/or hydrogen peroxide. In certain embodiments, the active agent comprises solid, gaseous or liquid iodine molecule with or without potassium iodine. In certain embodiments, additional active agent is incorporated in the web during the hydroentangling or calendaring step.

In yet another aspect, the invention is directed to a manufacturing process for producing an antitoxic nonwoven fabric comprising the steps of forming a plurality of staple fibers comprising a polymer and an iodinated resin, forming a web from said staple fibers via an air laid, wet laid or carded process, and subjecting said web to a hydroentangling or calendering process, wherein an additional active agent is incorporated into the web during said hydroentangling or calendering process, and isolating the fabric.

In still another aspect, the invention is directed to a manufacturing process for producing an antitoxic nonwoven fabric comprising the steps of forming a plurality of staple fibers comprising a polymer, forming a web from said staple fibers via an air laid, wet laid or carded process, and subjecting said web to a hydroentangling or calendering process, wherein an active agent is incorporated into the web during said hydroentangling or calendering process, and isolating the fabric.

In still another aspect, the invention is directed to a manufacturing process for producing an antitoxic nonwoven fabric comprising the steps of forming a plurality of staple fibers comprising a polymer, forming a web from said staple fibers via an air laid, wet laid or carded process, and subjecting said web to a hydroentangling or calendering process, wherein an active agent is incorporated into the web during said air laid, wet laid or carded process.

In yet another aspect, the invention is directed to a wound dressing comprising a hydroentangled wet laid nonwoven material, said nonwoven comprising polymer fibers embedded with iodinated resin powder. In particular embodiments, the hydroentangled wet laid nonwoven material can include an antitoxic agent in the liquid (for example: triiodide or triiodine). In certain embodiments, the dressing is manufactured using one of the aforementioned processes.

In still another aspect, the present invention is directed to a wipe or drape comprising a hydroentangled wet laid nonwoven material, said nonwoven comprising polymer fibers embedded with iodinated resin powder. In certain embodiments, the wipe and or drape is manufactured using one of the aforementioned processes.

In an additional aspect, the present invention is directed to a wipe, drape, gown, or air filter comprising a hydroentangled wet laid nonwoven material comprising an antitoxic agent (for example: triiodide or triiodine), said nonwoven comprising polymer fibers embedded with iodinated resin powder. In certain embodiments, the wipe and or drape is manufactured using one of the aforementioned processes.

In yet another aspect, the invention is directed to a wound dressing comprising an air laid and calendered nonwoven material followed by a single or multiple immersion in a liquid comprising an antitoxic agent in the liquid or gas for (for example: triiodide or triiodine) prior to being dried if desired, said nonwoven comprising polymer fibers embedded with or without iodinated resin powder. In certain embodiments, the dressing is manufactured using one of the aforementioned processes.

In still another aspect, the present invention is directed to a wipe, drape, gown, air filter, or non-woven comprising an air laid and carded nonwoven material comprising an antitoxic agent (for example: triiodide or triiodine) prior to being dried if desired, said nonwoven comprising polymer fibers embedded with or without iodinated resin powder. The non-woven can be submitted to a single or a multiple immersion in a liquid or gas comprising for example Triiodide or triiodine. In certain embodiments, non-woven is manufactured using one of the aforementioned processes.

In still another aspect of the present invention the fibers with or without iodinated resin particulates included are quenched or processed in a liquid or gas containing an antitoxic agent for example Triiodide or triiodine prior to, or after, being spooled in order to impart antitoxic properties to the fibers. These fibers can further be processed using one of the aforementioned processes for either a woven material or a non-woven material.

In another aspect, the invention is directed to a process for producing an antitoxic nonwoven fabric including: forming a plurality of staple fibers comprising a polymer; wet laying the staple fibers in an aqueous solution comprising an active agent selected from the group consisting of iodine, bromine, chlorine and hydrogen peroxide to generate a wet laid web; subjecting the wet laid web to hydroentangling or calendering; and isolating the fabric.

In yet another aspect, the invention is directed to a process for producing a biocidal nonwoven fabric including: forming a plurality of staple fibers comprising a polymer; air laying the staple fibers in a chamber containing a gaseous active agent selected from the group consisting of iodine, bromine, chlorine and hydrogen peroxide to generate a dry laid web; subjecting the dry laid web to hydroentangling or calendering; and isolating the fabric.

In still another aspect, the invention is directed to a process for producing a biocidal nonwoven fabric including: forming a plurality of staple fibers comprising a polymer; forming a web from the staple fibers via an air laid, wet laid, or carded process; subjecting the web to hydroentangling or calendering, wherein an active agent is incorporated into the web during the hydroentangling or calendering process, where the active agent includes one or more of iodine, bromine, chlorine and hydrogen peroxide; and isolating the fabric.

In another aspect, the invention is directed to a process for producing an antitoxic nonwoven fabric including: forming a plurality of staple fibers comprising a polymer and an antitoxic agent; wet laying the staple fibers in an aqueous solution comprising an active agent selected from the group consisting of iodine, bromine, chlorine and hydrogen peroxide to generate a wet laid web; subjecting the wet laid web to hydroentangling or calendering; and isolating the fabric.

In yet another aspect, the invention is directed to a process for producing a biocidal nonwoven fabric including: forming a plurality of staple fibers comprising a polymer and an antitoxic agent; air laying said staple fibers in a chamber containing a gaseous active agent selected from the group consisting of iodine, bromine, chlorine and hydrogen peroxide to generate a dry laid web; subjecting the dry laid web to hydroentangling or calendering; and isolating the fabric.

In yet still another aspect, the invention is directed to a process for producing a biocidal nonwoven fabric including: forming a plurality of staple fibers comprising a polymer and an antitoxic agent; forming a web from the staple fibers via an air laid, wet laid, or carded process; subjecting the web to hydroentangling or calendering, wherein an active agent is incorporated into the web during the hydroentangling or calendering process, where the active agent comprises one or more of iodine, bromine, chlorine and hydrogen peroxide; and isolating the fabric.

Another aspect includes any of the processes described herein further including incorporating an additional active agent in the web during the hydroentangling or calendering step.

The present invention is also directed to non-woven material prepared using any of the processes of the present invention described herein.

### DETAILED DESCRIPTION OF INVENTION

The following sections describe exemplary embodiments of the present invention. It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto.

Throughout the description, where items are described as having, including, or comprising one or more specific components, or where processes and methods are described as having, including, or comprising one or more specific steps, it is contemplated that, additionally, there are items of the present invention that consist essentially of, or consist of, the one or more recited components, and that there are processes and methods according to the present invention that consist essentially of, or consist of, the one or more recited processing steps.

It should be understood that the order of steps or order for performing certain actions is immaterial, as long as the invention remains operable. Moreover, two or more steps or actions may be conducted simultaneously.

Scale-up and/or scale-down of systems, processes, units, and/or methods disclosed herein may be performed by those of skill in the relevant art. Processes described herein are configured for batch operation, continuous operation, or semi-continuous operation.

The invention provides a novel method of making fabrics with antitoxic (e.g., biocidal) properties. The fabrics can be either wovens or nonwovens. The antitoxic properties are imparted to the fabric by introducing an active agent to the fabric, particularly an antimicrobial agent. The fabrics produced in accordance with the present invention have widespread utility. For instance, they can be used as wound dressings, non-wovens, gowns, drapes, air filters, protective clothing and wipes.

Antitoxic nonwoven fabrics produced in accordance with the present invention are produced using a multi-step process. The process begins with selecting a polymer or combination of polymers to spin into fibers. The selection of polymers will be dependent upon the desired application of the nonwoven fabric. Preferred polymers used in accordance with the present invention include but are not limited to polyamides, polyesters, polyolefins, copolymers of ethylene and propylene, copolymers of ethylene or propylene, terpolymers of ethylene with propylene, polylactic acid, ethylene vinyl acetate copolymers, propylene vinyl acetate copolymers, styrene-poly(ethylene-alpha-olefin) elastomers, polyurethanes, polyethers, polyether esters, polyacrylates, ethylene alkyl acrylates, polyisobutylene, polybutadiene, isobutylene-isoprene copolymers, and combinations of any of the foregoing. Particularly preferred polymers include polypropylene, polyethylene, PBT, nylon, alginate, polycarbonate, poly(4-methyl pentene-1) and polystyrene. Alternative substrates may further include glass fibers and fibers, such as cellulose.

To provide the desired antitoxic properties to the nonwoven fabric, an active agent, particularly an antitoxic agent, is selected. The antitoxic agent is preferably an antimicrobial agent, an antiviral agent, a bio-chemical agent or a reducing agent. The active agent preferably exerts a toxic effect on a diverse array of microorganisms and other pathogens and environmental toxins while not being toxic to the user. Preferably, the antitoxic agent comprises iodinated resin particles, triiodine or triiodide chemical. Other suitable active agents include but are not limited to triclosan, diatomic halogens, silver, copper, zeolyte with an antimicrobial attached thereto, halogenated resins, and agents capable of devitalizing/deactivating microorganisms/toxins that are known in the art, including for example activated carbon, other metals and other chemical compounds.

In accordance with preferred embodiments of the present invention, the chemical impregnation/incorporation of the active agent into the fabric is performed during multiple stages of the manufacturing process. For instance, chemical impregnation can occur during or immediately following fiber formation, during web formation and/or during post-treatment processes. It is found that the inventive methods provide for fabrics that display outstanding biocidal performance.

In one preferred embodiment of the present invention, the antimicrobial agent added to the nonwoven web is a demand disinfectant iodinated resin. U.S. Pat. No. 5,639,452 (“the '452 patent”) to Messier, the content of which is incorporated herein by reference in its entirety, discloses such a demand disinfectant resin and a process for producing the resin. The '452 patent further discloses that this disinfectant is a demand-type broad spectrum resin-polyiodide disinfectant useful in sterilizing fluids, and particularly a polyiodide disinfectant in which the iodine is more tenaciously associated with the resin than with previously known disinfectants, such that it leaves behind nondetectable or otherwise acceptable residual diatomic iodine in treated fluids. The demand disinfectant iodinated resins disclosed in the '452 patent are generally formed by contacting an anionic resin with an aqueous solution of iodine and potassium iodide under conditions of high temperature and pressure. Iodinated resin beads (TRIOSYN®) are made by Triosyn Research Inc., a division of Triosyn Corporation of Vermont, USA.

Despite the strength of the polymer resin/iodine bonds, it is found that iodine may be lost during fiber processing and web formation. As will become apparent from the disclosure below, a significant advantage of the methodology of the present invention is that iodine may be incorporated into the nonwoven material at various multiple stages of production. Hence, the inventive methods are capable of producing nonwovens that exert a toxic effect on a large array of microorganisms.

The iodinated resin may initially be incorporated into a nonwoven material by various methods, such as those described in U.S. Publication No. 2006/0144403. Preferably, the iodinated resin is incorporated directly into the fibers of the nonwoven material, thus forming an iodinated resin/polymer concentrate. The iodinated/resin/polymer concentrate is produced by compounding iodinated resin powder (micron size of approximate 1-10 μm) and polymer granules together. The size of the iodinated resin particles may be in the range of 2-30 μm inclusive, or 2-20 μm inclusive, or 5-15 μm inclusive. In one preferred embodiment, they are in the range of 5-10 μm, inclusive. The compounding process may consist of mixing the iodinated resin powder and the polymer granules together in a container and/or mixer to distribute the particles properly in the batch. This mixture is then poured into the hopper (reservoir) of the compounding system. The mixture slowly goes from the hopper and into the compounder through, for example, 5 different heat zones to melt the solids together. The iodinated resin containing polymers are then spun and the resultant filaments are extruded through a dye-tip. The iodinated resin powder can also be inserted separately than the polymeric granules and at a different heat zone.

Following extrusion of the spun filaments with incorporated iodinated resin, the filaments can be wound together to produce a single fiber or alternatively, the filaments could have been initially extruded in order to generate one or more single strands. The fiber may have a lubricant finish added onto its surface. During this step an antitoxic agent can be added. In one embodiment, the antitoxic agent can be added during this step to spun filament formed without the step of incorporating iodinated resin. Subsequently, the fiber is stretched and wound onto a cardboard bobbin. Fibers produced from the above-described methodology are generally wound on each bobbin at a certain rate and at different stretching pressures to produce fibers of different diameters. A specific weight per bobbin is produced to match what is needed to cut the fiber into staple fibers of the needed length for the desired application, with and without crimping. To make crimped fibers, fibers from several equal weight bobbins are fed onto a crimp system. According to certain embodiments for nonwoven carding processes (discussed in more detail hereinbelow), it is found that the desired number of crimps is 10-20 crimps per inch and the optimal length of the fiber is between 1-2 inches in length. The resultant fibers (crimped or straight) are fed onto a cutter to cut the fibers into staple fibers. The desired lengths vary depending on the nonwoven process used in the following step but generally range in size from about 0.5 inches to about 2.0 inches.

Following formation of the staple fibers containing the antitoxin agent, which can be iodinated resin, the nonwoven web is formed. Formation of the nonwoven web may be accomplished through a wet laying procedure using iodinated resin-containing staple fibers. The staple fibers may be produced from a single polymer of a particular diameter or from a single polymer of varying diameters produced by methods described above. Alternatively, antitoxin agent-containing fibers made from different polymers may be used to form the nonwoven web. It is often advantageous to include structural fibers and/or absorbent fibers and/or binders (further referred to as absorbant fibers) to impart desired properties to the nonwoven material. In such cases, cut fibers are weighed out to the desired concentration of (a) an antitoxic agent-containing, preferably iodinated resin-containing, fibers, (b) absorbent fibers, and/or (c) binders to generate a composite blend. The ratio of these components will vary based on the desired application of the material. Antitoxic-, for example, iodinated resin-containing fibers prepared from different polymers may be used in the nonwoven blend. The amount of antitoxic agent may vary from 0.2% to 90% by weight, preferably from 0.2% to 25%. For example, iodinated resin-containing fibers may vary from about 5% to 100% of the composite blend, preferably from about 30% to about 80% of the composite blend and most preferably from about 50% to about 70% of the composite blend. The amount of adsorbent fibers may vary from about 0.1% to about 95% of the composite blend, preferably from about 20% to about 40% of the composite blend, and most preferably from about 30% to about 40% of the composite blend. Preferred adsorbent fibers include but are not limited to rayon, alginate, cellulose pulp and cellulose acetate. The amount of binder may vary in content from about 0% to about 25% with fibers of different lengths and deniers. Preferred binders from MINIFIBERS Inc, Fiber Innovation Technologies, and/or other companies of the sort, may include the following combinations of bicomponent fibers: high density polyethylene (HDPE)/polypropylene (PP), HDPE/polyester, Bionelle/Biomax Aliphatic PET, Bionelle/PolyLactic Acid (PLA), Co-Polyester/Polyester, Co-Polypropylene/PP, and Ethyl Vinyl Acetate/PP, PLA/PLA.

In the wet laying process, the iodinated resin containing fiber, optionally adsorbent fibers, and optionally binder are mixed using a blender to make sure the fibers are uniform. Wet laid processes generally use water. We observed that a quantity of iodine is lost from the iodinated resin containing fibers after the staple fibers are added to water. We found it is possible to overcome this problem by adding iodine molecule to the aqueous solution prior to adding the staple fibers. The iodine is preferably added at a high concentration, close to saturation (e.g., 300 ppm to 5000 ppm by mixing iodine and potassium iodide). The iodine molecule may be added with or without potassium iodide to the water or any other solvent. Potassium iodide assists in converting the diatomic iodine to triiodide ions. When the staple fibers are added to the iodine solution, iodine can then incorporate into the active sites of the iodinated resin. Additionally, the iodine may insert into the other fibers of the composite blend including the absorbent fibers and the binder. This procedure not only minimizes iodine loss associated with the wet laying process and the preceding procedures but significantly increases the amount of iodine in the nonwoven web. Accordingly, the procedure of forming staple fibers followed by wet laying in an aqueous iodine solution results in nonwoven webs with greater iodine content, and thus greater efficacy, than previously described meltblown processes.

In addition to iodine, other active agents may be added to the aqueous solution prior to the wet laying process. Some preferred molecules include bromine, chlorine, fluorine and hydrogen peroxide. In alternative embodiments, the aqueous solution comprises ethanol, 1-propanol, 2-propanol, isopropanol, cationic surfactant (e.g., benzalkonium chloride, chlorhexidine, octenidine dihydrochloride), metals, a quaternary ammonium compound (e.g., benzalkonium chloride (BAC), cetyl trimethylammonium bromide (CTMB), cetylpyridinium chloride (Cetrim, CPC), benzethonium chloride (BZT), chlorhexidine, octenidine), boric acid, brilliant green, chlorhexidine gluconate, mercurochrome, manuka honey, octenidine dihydrochloride, phenol (carbolic acid), sodium chloride, sodium hypochlorite, calcium hypochlorite, terpenes, and/or poly-hexa-methyl-biguanide (PHMB). These active agents can be added alone or in combination with iodine molecule depending on the desired performance of the ultimate nonwoven material.

After adding the antitoxic containing fibers containing, for example, iodinated resin, Triiodine, or Triiodide, and other fibers to the iodine (or other active agent) solution to form a slurry, the resultant mixture is pressurized under vacuum or pressure. A thin sheet of loosely bound material is then isolated from the aqueous medium. The sheet is heated in an oven, for example, in a static or continuous thermal process at the minimum temperature that will melt the binder and the fibers and give shape to the newly formed wet laid web. The wet laid web serves as a precursor for subsequent processing such as hydroentanglement or calendering.

As discussed in the Background Section, dry forming processes such as air laying or carding may be used rather than wet laying to form the nonwoven filter media. The air laid process is similar to the wet laid in the preparation of the fibers and the mixture in the blender system. The mixture is then inserted on top of a column through a system that pushes air through the large column. Once the total amount is inserted, the system is shutdown and recuperated on the bottom screen. As with the wet laying process described above, the dry forming process presents an opportunity to add iodine and/or other active agents to the forming web during the process. In the air laying process, the molecule is sublimed in a chamber where the fibers or media will pass, hence imparting the desired microbicidal properties to the web. After the newly formed web passes through the chamber, a thin sheet of loosely bound fibers is isolated. The sheet is then placed in an oven at the minimum temperature that will melt the binder and the fibers and give shape to the sheet. The dry laid web serves as a precursor for subsequent processing such as hydroentanglement or calendering.

Alternatively, the carding process relies on an instrument that contain numerous rollers, preferably 8 or more that will take the mixed fibers and will have them processed through to give a layer of nonwoven media of a given basis weight. The system may contain a section at the end where the media could pass through an aqueous solution of iodine or iodine/potassium iodide. Hence, the iodine becomes impregnated in the fibers comprising the composite blend at varying contact times before it passes through a series of rollers that will remove the excess liquid and begin drying the resultant web. Similar to the wet laying and dry forming processes described above, the methodology provides a means of recovering any iodide from previous processing steps. The carded web serves as a precursor for subsequent processing such as hydroentanglement or calendering.

Following web formation, the wet laid, air laid or carded webs may be subjected to further processing steps. The post-treatment processes give the product the desired properties such as strength, flexibility, etc. One preferred post-treatment method is hydroentanglement, also referred to as spunlacing. Hydroentanglement is a process for forming a fabric by mechanically wrapping and knotting fibers in a web through the use of high-velocity jets of water. In a conventional hydroentangling process, the large amounts of water and high pressures required may have a detrimental effect on the antitoxic (e.g., microbicidal) performance of the web because the iodine may dissociate from the fibers to a significant degree. We have found that rather than using pure water during the process, a solution containing iodine molecule, with or without potassium iodide, can be used instead. The fabrics produced have the desired strength and other beneficial properties. Moreover, owing to the presence of the large quantities of iodine molecule in the web, the fabrics produced by this method show outstanding biocidal performance. Although the hydroentangling method has been described using a solution of iodine at high pressure, other molecules can be used in place of or in addition to iodine. Such molecules include but are not limited to bromine, chlorine, fluorine, hydrogen peroxide, for example.

The hydroentangled fabrics produced by the methods of the present invention have desirable properties including softness, high drape and comfort. Owing to the large range of fibers that can be employed and the broad range of process variables, the products produced by the method are quite versatile. For instance, products including, but not limited to wound dressings, gowns, drapes, air filters, clothing and wipes (all types) can be produced in accordance with the methods of the present invention. The products have the significant advantage of showing strong levels of protection against microbes and other harmful agents.

Another post-process process that can be used is calendering. The process uses two hot rollers (at varying heat and pressure) to make the nonwoven media thinner as well as to give the media more structure and a pattern. As with the hydroentanglement process, the wet laid, air laid and carded webs serve as precursors for the calendering process. Calendering may have the effect of losing iodine as the material passes through the rollers. To mitigate this effect, the fabric leaving the rollers may be passed into a solution of iodine to help recharge the sites and give the iodine load that is required.

In addition, the calendaring can be performed in a wet environment where the calendaring rollers are either fully submerged or in a shower type environment while processing the media that must be calendered. This process is applicable to the production of all nonwoven fabrics.

### EXAMPLES

The following examples demonstrate that the physical location of particulates of anionic iodinates resin (TRIOSYN®) on a non woven and/or woven media combined with a post treatment of this same media with an iodinated solution is found to have an important and scientifically significant difference on its effectiveness to kill a broad spectrum of tough microorganisms in a short lapse of time, as well as on the amount of iodine that is released by the non woven media over an 8 hour period. In these examples, distinctively different processing technologies for combining TRIOSYN® with a material are compared:
Method A: Using a glue (adhesive) to stick the TRIOSYN® anti-microbial powder on the surface of a non woven.
Method B: Integrating TRIOSYN® powder in a polymer and co-extruding it into a fiber which is carded into a non woven media having been submitted to a single triiodide treatment during hydroentanglement.
Method C: Integrating TRIOSYN® powder in a polymer and co-extruding it into a fiber which is carded into a non woven media having been submitted to a first triiodide treatment during hydroentanglement and then doing a secondary post-treatment with tri-iodide 1500 ppm.

### 1.0 Description of AATCC Testing Method

The test method used to evaluate antimicrobial performance in microbiology is the AATCC100 (2004 modified) method. This method is used herein to assess the effectiveness of TRIOSYN® hydrophobic materials in devitalizing bacteria. This method essentially provides a quantitative procedure for the evaluation of the degree of antimicrobial activity intended in the use of such materials.

### 1.1 Pseudomonas Aeruginosa Challenge

The antimicrobial performance of two conceptually different types of media was challenged with pseudomonas aeruginosa bacteria. The first type of media was produced according to Method A described briefly above, and contained TRIOSYN® particles glued at the surface of a non woven media. The second type of media was produced according to Method B described briefly above, and consisted of TRIOSYN® particles co-extruded inside each fiber of Polypropylene. These fibers were then carded into a non woven material.

The AATCC100 (2004 modified) procedure for testing the effectiveness of these media in devitalizing bacteria is broken down into the following steps:
Prepare a stock of desired microorganism, spike a nutrient broth (or TSB) and incubate for 18 hours at 37° C. in shaking incubator set at 200 RPM.
Centrifuge at 2200 RPM the bacterial culture and adjust the absorbance with PBS to approximately 0.900 at 600 nm (approximately 2.0⁰⁸ CFU/mL)
Seed 10 mL of molten agar slurry (kept in a water bath at 55° C.) with 1 mL of 10⁰⁸ CFU/mL challenge suspension
Individually place the treated and untreated samples in sterile 100 mm Petri dishes on top of a clean microscope slide.
Inoculate the swatches with 0.1 ml of the challenge suspension and spread it with a sterile glass bent rod (if the material is very hydrophobic, let the agar slurry cool to 40° C.).
Let stand in the Petri dishes at room temperature.
After the contact periods, aseptically transfer microscope slide-material in a 50 mL centrifuge tube containing 10 mL of Neutralizing solution (PBS/0.5% Polysorbate 80/0.1% Thiosulfate or NDS)
Vortex the samples for several seconds.
Serially dilute in PBS (dilutions of 10E-00, 10E-01 and 10E-02 are usually suitable for the treated samples; 10E-02, 10E-03 and 10E-04 are usually suitable for untreated samples) and plate on TSA.
Incubate microorganism at 37.5° C.±2° C. for 24-48 hours.

### 1.1.1 Results for Pseudo Log Reduction (Fresh Samples)

### Test Details:

Microbiological (AATCC100 v3.2) stability performance of fresh nonwoven hydrophobic materials (samples stored at room temperature)
Challenge organism: Pseudomonas Aeruginosa (in 0.3% agar slurry)
Contact Time: 15 min
Neutralization: 10 ml PBS-TT
   Results are provided in cfu/mL (“colony forming units”/mL), which represents the amount of bacterial colonies per milliliter of sample.

TABLE #1
Antimicrobial Performance of Fresh Samples

| | | Blank | TRIOSYN ®-coated | |
|---|---|---|---|---|
| | Contact | Control | | Log |
| | Time | (CFU | (CFU | Reduc- |
| Sample Description | (min) | Total) | Total) | tion |
| Media 1 (Method A): | 15 | 5.22E+06 | 2.00E+02 | 4.42 |
| TRIOSYN ® particles | | | | |
| glued at the surface | | | | |
| of the media | | | | |
| Media 2 (Method B): | 15 | 7.38E+06 | <5.00E+01 | >5.17* |
| TRIOSYN ® particles | | | | |
| co-extruded inside | | | | |
| each fiber with single | | | | |
| Triiodide treatment | | | | |

*BDL: Below the detection limit

As newly made fresh media, the two different concept Medias perform very well. However, of the two, media 2 with the TRIOSYN® in the fiber clearly showed superior performance.

### 1.1.2 Results for Pseudo Log Reduction (Aged Samples)

### Test Details:

Microbiological (AATCC100 v3.2) stability performance of aged nonwoven hydrophobic materials (samples stored at higher temperatures)
Challenge organism: Pseudomonas Aeruginosa (in 0.3% agar slurry)
Contact Time: 15 min
Neutralization: 10 ml PBS-TT

TABLE #2
Antimicrobial Performance of Aged Samples

| | | Contact | Blank | TRIOSYN ®-coated | |
|---|---|---|---|---|---|
| | Sample | Time | Control | (CFU | Log |
| Sample Description | Storage | (min) | (CFU Total) | Total) | Reduction |
| Media 1 (Method A): | Incubated for | 15 | 6.87E+06 | 5.78E+03 | 3.07 |
| TRIOSYN ® particles | 2 weeks | | | | |
| glued at the surface | at 45° C. | | | | |
| of the media | | | | | |
| Media 2 (Method B): | Incubated for | 15 | 2.48E+06 | <5.00E+01 | >4.70* |
| TRIOSYN ® particles | 2 weeks | | | | |
| co-extruded inside | at 50° C. | | | | |
| each fiber with single | | | | | |
| Triiodide treatment | | | | | |

*BDL: Below the detection limit

After aging both media in an oven at 45 C or 50 C for 2 weeks, a difference in performance between media 1 and 2 can be seen to increase when compared to the fresh results from Table #1. Media 2, having anionic iodinated resin extruded within the fibers, is performing on par with the same non-aged sample shown in Table #1, while the performance of Media 1, which has particulates glued to the surface, has degraded substantially.

### 1.1.3 Results for Pseudo Log Reduction (Post Simulated Breathing Treatment)

### Test Details:

Microbiological (AATCC100 v3.2) stability performance of nonwoven hydrophobic materials (samples after breathing machine for 5 hours): Media 1 and 2 of Table #3 have same amount of TRIOSYN® as the corresponding fresh samples in Table #1
Test Set-up: Internally developed breathing machine (10LPM breathing rate; inhaled air at ambient conditions; exhaled air at 85% RH and 38° C.)
Challenge organism: Pseudomonas Aeruginosa (in 0.3% agar slurry)
Contact Time: 15 min
Neutralization: 10 ml PBS-TT

TABLE #3
Antimicrobial Performance of Samples after Breathing Machine for 5 Hours

| | | Contact | Blank | TRIOSYN ®-coated | |
|---|---|---|---|---|---|
| | Sample | Time | Control | (CFU | Log |
| Sample Description | Treatment | (min) | (CFU Total) | Total) | Reduction |
| Media 1 (Method A): | After 5 hours | 15 | 8.57E+06 | 7.39E+03 | 0.06 |
| TRIOSYN ® particles | breathing | | | | |
| glued at the surface | machine | | | | |
| of the media | | | | | |
| Media 2 (Method B): | After 5 hours | 15 | 2.00E+06 | <5.00E+01 | >4.57* |
| TRIOSYN ® particles | breathing | | | | |
| co-extruded inside | machine | | | | |
| each fiber with single | | | | | |
| Triiodide treatment | | | | | |

*BDL: Below the detection limit

After passing air through both media candidates for 5 hours, simulating normal human usage, Media 1, with the anionic iodinated (TRIOSYN®) particles glued onto the surface, has lost its microbiocidal performance when compared to the same media before use (Table #1). The media with TRIOSYN® integrated into the fibers has retained its full killing performances of microorganisms after being challenged with air for 5 hours when compared to a non used sample produced by the same Method and with the same amount of TRIOSYN®.

### 1.2 Clostridium Difficile (C. Difficile) Challenge

The antimicrobial performance of two conceptually different types of media was challenged with clostridium difficile bacteria. Results are shown in Table #4 below. The first media tested (Media 2) was produced according to Method B and contained anionic iodinated (TRIOSYN®) particles extruded within the fibers of the non woven media. The second media tested (Media 3) was produced according to Method C and consisted of anionic iodinated (TRIOSYN®) particles co-extruded inside each fiber of Polypropylene with a post treatment of iodine re-impregnation. These fibers were then carded into a non woven material.

The AATCC100 (2004 modified) method procedure used to produce the results shown in Table #4 below is as follows:
Prepare a stock of desired microorganism from spores previously prepared, spike a nutrient broth (or TSB) and incubate for 18 hours at 37° C. in shaking incubator set at 200 RPM.
Seed 10 mL of molten agar slurry (kept in a water bath at 55° C.) with 1 mL of 10⁰⁸ CFU/mL challenge suspension
Individually place the treated and untreated samples in sterile 100 mm Petri dishes on top of a clean microscope slide.
Inoculate the swatches with 0.1 ml of the challenge suspension and spread it with a sterile glass bent rod (if the material is very hydrophobic, let the agar slurry cool to 40° C.).
Let stand in the Petri dishes at room temperature.
After the contact periods, aseptically transfer microscope slide-material in a 50 mL centrifuge tube containing 10 mL of Neutralizing solution (PBS/0.5% Polysorbate 80/0.1% Thiosulfate or NDS)
Vortex the samples for several seconds.
Serially dilute in PBS (dilutions of 10E-00, 10E-01 and 10E-02 are usually suitable for the treated samples; 10E-02, 10E-03 and 10E-04 are usually suitable for untreated samples) and plate on TSA.
Incubate at microorganism 37.5° C.±2° C. for 24-48 hours.

### 1.2.1 Results for C. Difficile Log Reduction

### Test Details:

Microbiological (AATCC100 v3.2) stability performance of nonwoven hydrophobic materials (samples stored at room temperature)
Challenge organism: Clostridium Difficile (in 0.3% agar slurry)
Contact Time: 15 min
Neutralization: 10 ml PBS-TT

TABLE #4
Antimicrobial Performance of Samples (C. Difficile challenge)

| | | Blank | TRIOSYN ®-coated | |
|---|---|---|---|---|
| | Contact | Control | | Log |
| | Time | (CFU | (CFU | Reduc- |
| Sample Description | (min) | Total) | Total) | tion |
| Media 2 (Method B): | 15 | 3.88E+04 | 3.30E+03 | 1.07 |
| TRIOSYN ® particles | 30 | 3.10E+04 | 4.40E+03 | 0.85 |
| co-extruded inside | | | | |
| each fiber with single | | | | |
| triiodide treatment | | | | |
| Media 3 (Method C, I₃⁻ | 15 | 3.88E+04 | 5.00E+02 | 1.89 |
| post-treated): | 30 | 3.10E+04 | 6.67E+01 | >2.67* |
| -TRIOSYN ® particles | | | | |
| co-extruded inside | | | | |
| each fiber with first | | | | |
| triiodide treatment | | | | |
| followed by a | | | | |
| -Post treatment with | | | | |
| 1500 ppm I₃⁻ | | | | |

*BDL: Below the detection Limit

The challenge with C. Difficile spores is much more challenging than with Pseudomonas. Tests were performed on the Media 2 with TRIOSYN® co-extruded, and on the same media with a post-treatment in a solution of Tri-iodide, labeled Media 3 in Table #4 above. The results show that Media 2 kills less than 1 log at 30 min. Media 3, which is the same as Media 2 but with an additional post-treatment, kills all of the C. Difficile at 30 min.

### 1.3 Toxicology Air Testing

Toxicology air testing of three types of media was performed to determine the Iodine release levels. The first type of media was the one glued with anionic iodinated (TRIOSYN®) particles at the surface of the media (Media 1), and the second type was media consisting of anionic iodinated (TRIOSYN®) particles co-extruded inside each fiber (Media 2). The third type was the same as the second sample, but post treated with 1500 ppm tri-iodide solution for 15 minutes (Media 3).

Toxicology air testing was performed using a glass impinger containing 10 mL of sodium carbonate trapping solution, at a constant flow for a period of 15 minutes. The sodium carbonate solution absorbs any Iodine present in the airstream, and the exact concentration of Iodine is determined by ion chromatography (HPLC), which measures Iodine in the form of Iodide (I⁻). Importantly, the detection limit of Iodide is 0.0010 ppm since this is the lowest Iodide standard measured by the HPLC method.

### 1.3.1 Toxicology Air Testing of Fresh Samples (Chemistry)

The samples listed in Table #5 below were air tested and analyzed by HPLC for Iodine release.

TABLE #5
Toxicology Air Testing Results for Media

| | Iodine Concentration (mg/m³) | | |
|---|---|---|---|
| | | Media 2 (Method B): | Media 3 (I₃⁻ treated-Method C): |
| | Media 1 (Method A): | TRIOSYN ® particles | -TRIOSYN ® particles co-extruded |
| Time | TRIOSYN ® | co-extruded inside | inside each fiber with a single |
| Point | particles glued at the | each fiber with a | triiodide treatment followed by a |
| (min) | surface of the media | single triiodide treatment | -Post treatment with 1500 ppm I₃⁻ |
| 15 | 0.5934 | 0.0117 | 0.0294 |
| 30 | 0.4955 | 0.0103 | 0.0304 |
| 45 | 0.3957 | 0.0064 | 0.0234 |
| 60 | 0.3508 | 0.0043 | 0.0241 |
| 75 | 0.2790 | 0.0034 | 0.0199 |
| 90 | 0.2517 | 0.0030 | 0.0210 |
| 105 | 0.2235 | 0.0030 | 0.0193 |
| 120 | 0.2037 | 0.0027 | 0.0172 |
| 135 | 0.1858 | 0.0045 | 0.0186 |
| 150 | 0.1806 | 0.0019 | 0.0156 |
| 165 | 0.1788 | 0.0021 | 0.0112 |
| 180 | 0.1737 | 0.0009* | 0.0111 |
| 195 | 0.1776 | 0.0012 | 0.0097 |
| 210 | 0.1659 | 0.0009 | 0.0106 |
| 225 | 0.1553 | 0.0015 | 0.0096 |
| 240 | 0.1435 | 0.0010 | 0.0099 |
| Dietary Intake | 2163.98 | 28.29 | 206.25 |
| for 8 hrs = | | | |

Lowest standard measured at 0.0010 ppm iodide
*BDL = Measured Concentration of Iodide less than 0.0010 ppm (equal to 2.0 × E−03 mg/m3 iodine)

The iodine toxicology results clearly show that the anionic iodinated (TRIOSYN®) glued on the surface (Media 1) releases 77 times more iodine than when the TRIOSYN® is in the fiber (Media 2). For Media 3, i.e., when the post-treatment is used on Media 2, the iodine released is 10 times more than the sample, Media 2, for example, at 240 minutes with no pre-treatment. However, the amount of iodine released at the same time is still 15 times lower than that released by Media 1. Moreover, as shown in Table #4, Media 3 shows significantly better performance in killing C. Difficile spores than either Media 1 or Media 2. These results confirm that Media 3 shows a superior combination of 1) effectiveness in devitalizing a wide spectrum of tough microorganisms over a short lapse of time and 2) low toxicity levels.

Although the above embodiments have been described for iodinated resin-containing fabrics, the methodology applies to other antimicrobial agents as well. Furthermore, the active agent may be added at any step of the process. For instance, in the methods described above, iodine was introduced into the melt prior to fiber formation and it was also introduced in later steps of the process such as wet laying and hydroentangling. Based on the desired performance characteristics of the fabric, it may be only necessary to introduce the active agent at a single step in the process. For instance, in alternative embodiments, the active agent may be introduced for the first time during the wet laying process only. In such cases, the staple fibers would be produced as described above but without any antimicrobial agent incorporated therein. The resultant fibers will then be subjected to a solution containing an active agent such as a halogen or hydrogen peroxide. Moreover, the active agent could be introduced at the hydroentangling or calendering stage in addition to or alternatively to incorporating during the wet laying, dry laying, or carding process.

The methodology above can also be applied to the production of woven fabrics. Similar to the production of nonwovens, filaments with an active agent incorporated would be produced and wound on a fiber bobbin. The fibers can then be intertwined with other fibers to generate a woven material.

The methodologies above can also be applied to already physically formed non-woven example: melt blown, spunbond, or other, where the finish nonwoven material can be immerse in a gas or liquid of the antitoxic agent either one or a multiple times prior to a drying process in order to impart the desired properties.

1-3. (canceled)

4. The process of claim 7, wherein an additional active agent is incorporated in the web during the hydroentangling or calendering step.

5. The process of claim 7, wherein the polymer comprises one or more members selected from the group consisting of polypropylene, polyethylene, nylon, polycarbonate, poly(4-methyl pentene-1), polystyrene and cellulose acetate.

6. The process of claim 7, wherein said wet laid web further comprises adsorbent fibers of one or more types selected from the group consisting of rayon, alginate, cellulose pulp and cellulose acetate.

7. A process for producing an antitoxic nonwoven fabric comprising:
a. forming a plurality of staple fibers comprising a polymer;
b. wet laying said staple fibers in an aqueous solution comprising an active agent selected from the group consisting of iodine, bromine, chlorine and hydrogen peroxide to generate a wet laid web;
c. subjecting said wet laid web to hydroentangling or calendering; and
d. isolating the fabric.

8. A process for producing an antitoxic nonwoven fabric comprising:
a. forming a plurality of staple fibers comprising a polymer;
b. air laying said staple fibers in a chamber containing a gaseous active agent selected from the group consisting of iodine, bromine, chlorine and hydrogen peroxide to generate a dry laid web;
c. subjecting said dry laid web to hydroentangling or calendering; and
d. isolating the fabric.

9. A process for producing an antitoxic nonwoven fabric comprising:
a. forming a plurality of staple fibers comprising a polymer;
b. forming a web from said staple fibers via an air laid, wet laid, or carded process;
c. subjecting said web to hydroentangling or calendering, wherein an active agent is incorporated into said web during said hydroentangling or calendering process, said active agent comprising iodine, bromine, chlorine and hydrogen peroxide; and
d. isolating the fabric.

10. The process of claim 7, wherein (a) step further comprises forming the plurality of staple fibers comprising the polymer and an antitoxic agent.

11-13. (canceled)

14. The process of claim 10, wherein the antitoxic agent is a biocidal agent.

15. A non-woven material prepared using the process of claim 4.

16. (canceled)

17. A wound dressing comprising the non-woven material of claim 15.

18. The process of claim 14, wherein the biocidal agent is an iodinated resin.

19. A non-woven material prepared using the process of claim 5.

20. A non-woven material prepared using the process of claim 6.

21. A wound dressing comprising the non-woven material of claim 19.

22. A wound dressing comprising the non-woven material of claim 20.
